(19) United States
(12) Patent Application Publication
(10) Pub. No.: US 2023/0255571 A1
(43) Pub. Date: Aug. 17, 2023

(54) A SYSTEM, AND METHOD TO PROVIDE TELEPRESENCE ROBOT EYE EXAMINATION SERVICES VIA MAIN CONTROL BASE, IOT, AND WIRELESS NETWORKS.

(71) Applicant: WILLIS DENNIS GRAJALES, Little Elm, TX (US)

(72) Inventor: WILLIS DENNIS GRAJALES, Little Elm, TX (US)

(21) Appl. No.: 17/672,692

(22) Filed: Feb. 16, 2022

(51) Int. Cl. A61B 5/00; H04W 4/024; H04W 4/30; G05D 1/02; A61B 3/028; A61B 3/103; A61B 3/032

(52) U.S. Cl. CPC A61B 5/7465; H04W 4/024; H04W 4/30; G05D 1/0276; A61B 3/0285; A61B 3/103; A61B 3/032

(57) ABSTRACT

System, methods and apparatus for providing telepresence robot vision screenings, and eye examinations via at least one of a plurality of telepresence robot eye examination systems anywhere in the world. A telepresence robot enhanced and adapted with at least one electronic device configured to be connected to at least one eye examination equipments of any maker. Whereby a human controller with a electronic device running mobile application and/or softwares can remotely control the robot to navigate and perform an indirect to direct eye examination and vision screening to at least one user located near the telepresence robot location. The system internet connection via wifi, bluetooth and/or cellular wireless network connection (4G, 5G and/or greater) to receive a connection from a controller. A eye examination, vision screening and/or medical referral is provided to at least one user via remote printing device and/or encrypted email.

### RELATED APPLICATIONS

The present application is a continuation of U.S. patent application Ser. No. 17/352,329 filed on Jun. 20, 2021. The present application is a continuation of U.S. patent application Ser. No. 17/307,901 filed on May 4, 2021. The present application is a continuation of U.S. patent application Ser. No. 17/180,789 filed on Feb. 20, 2021, which claims benefits of U.S. Provisional Patent Application No. 62/982,040 filed Feb. 26, 2020. The entire applications are incorporated herein by this reference.

### FIELD OF INVENTION

The present disclosure relates to a system and method for providing eye health, eye examinations, and vision screenings via a telepresence robot coupled to an MCB and equipment. The telepresence robot and its parts can be controlled by a remote controller where at least one remote controller from a network of the controller may be a health care provider, technician, and/or assistant. At least one remote controller operating at least one portable electronic device running a mobile application from anywhere in the world to provide eye exam services to at least one user in the same, near, remote, and/or distance geographic location. The interconnection from the remote controller and the telepresence robot system and its parts is achieved via wireless remote administration tool technology and/or remote access software, emulating software, screen mirroring, screen sharing, and/or screen control software.

The communication protocol used to achieve the interconnection from the telepresence robot system and the remote controllers portable electronic device are achieved via internet, cellular wireless networks, bluetooth, wifi and/or satellite networks. The coupling interconnecting telepresence robot, MCB, ophthalmic equipment and medical equipment comprising wired, bluetooth, wifi, zig-bee, wave, matter, cellular wireless networks and/or any other radio frequency telecommunication system.

### SUMMARY

Disclosed is a system and method for providing eye examination and vision screening to at least one user using a telepresence robot system and/or a modular MCB system. The telepresence robot is configured to be remotely controlled by a remote controller with a portable electronic device from any distance. At least one remote controller may be a health care provider, technician, or assistant. Where when referring to a health care provider, we mean any health care provider, including an optometrist ophthalmologist.

The remote controller portable electronic devices PED may be any electronic device running at least one mobile application. The portable electronic device may be a virtual reality headset, laptop, smartphone, smartwatch, and/or augmented reality headset. At least one mobile application or software on the portable electronic device may function as a remote administration tool (RAT) and/or remote access software (RAS). The RAT or RAS software is configured to be used to remote control the telepresence robot setup and the MCB.

The remote controller's portable electronic devices run a mobile application. The mobile application or software enables secure interconnections between the remote controller device and the telepresence robot system. The telepresence robot is coupled with a computer system called the main control base smart hub to form the system. The interconnection from the remote controller to the telepresence robot system is achieved via wireless networks such as wifi, cellular wireless networks (4G, 5G, and/or greater), and/or satellite internet networks. We call the system a telepresence robot eye examination system TREES. The following TREES describes a modular system that integrates at least one MCB, nondigital equipment, and/or digital ophthalmic equipment (EES). The system is further configured to adapt and to have removable components equipment modules reconfigured to be interchanged if needed to adapt to diverse applications.

The TREES can interconnect to at least one remote controller via remote administration tool and/or remote access software. The remote controller utilizes a portable electronic device running a mobile application that works as a remote administration tool and/or remote access software. The mobile application interconnects to the TREES to provide remote controllers the capabilities of providing indirect to direct and/or direct to direct eye examination. The TREES have video audio conference capabilities and can run mobile applications and software for text to speech, speech to text, vision acuities.

The remote controller can control the TREES that can control handheld auto refractor or an autonomous auto-refractor device to perform objective refraction to both eyes of a user. The TREES also includes ocular health cameras that can be used for the remote controller to view and control. The TREES also has capabilities of controlling subjective refractor software that controls a digital phoropter, variable lens phoropter, digital optometer and/or digital focometer. The remote controller can control the TREES to perform subjective refraction to a user both eyes from anywhere in the world.

### BRIEF DESCRIPTION OF THE DRAWINGS

FIG. 1 illustrates an embodiment of a telepresence robot eye examination system TREES and multiple first computer device with control softwares and equipment connected to a first computer device called the MCB. Where a remote controller with portable electronic devices is interconnected via the internet network to achieve remote control, screen view and screen control connection with the MCB, telepresence robot device. Where virtual reality headset running virtual reality software can also be seen connected to MCB.

FIG. 2, is an illustration of a pictorial diagram of a method of using a telepresence robot eye examination system.

FIG. 3 illustrates of a pictorial diagram of at least one form of user interface of the MCB that is also screen mirrored onto the remote controllers PED user interface. The MCB dashboard configured to function as a smart medical hub with at other EES computer system control softwares emulated onto the graphical user interface.

FIG. 4 illustrates a pictorial diagram of an embodiment the possible modular telepresence robot eye examination setup configurations. Where the second electronic device may be Laptop, Tablet, telepresence robot, augmented reality headset, and/or virtual reality headset. Where also that a EES may also be a digital telepresence robot, digital pupillary distance meter, digital lens meter, digital subjective refractor, digital auto-refractor, digital optometry unit table, virtual reality headset, and/or augmented reality headset. Where additional equipment E2 that may be connected to the second electronic device MCB may be portable printer, eye tracking hardware, and/or iris-scope.

FIG. 5 illustrates a pictorial diagram of an embodiment of the telepresence robot eye examination system. The TREES coupled, connected and/or pared too at least one main control base. The main control base being a second electronic device running at least one softwares to emulate at least one first electronic device equipment control softwares and coupled, connected and/or paired via wired, zigbee, z-wave, thread, matter, bluetooth, cellular wireless networks, and/or wifi to a medical, ophthalmic, temperature checking, virtual reality, augmented reality and/or eye examination equipment. Where the MCB and/or the Telepresence electronic device display screen display and run vision chart softwares.

FIG. 6 illustrates an embodiment of a third electronic devices running mobile application, browser and/or software. Where the controllers third electronic device TED can be a virtual reality head set, augmented reality headset, smartwatch, laptop, and/or tablet. Where via a mobile application a remote controller can sign up to become a provider of remote control telepresence robot eye examination services and vision screening services.

FIG. 7 illustrates different embodiments of possible controllers third electronic devices running mobile application, browser and/or software. Where the controllers can be sign in to become a controller via crypto wallet and/or regular signing secure connection. The controller is able to view the scheduling data from health care booking servicer and/or from a MCB interconnection.

FIG. 8 illustrates an embodiment of a diagram of how at least one of a plurality of controller from anywhere in the world can connect via the mobile application network can provide telepresence robot and/or MCC eye examination and vision screening services to a one of a plurality of location in the world via the system.

FIG. 9 illustrates an embodiment of a TREES being controlled by a remote controller using a PED and mobile application interconnect to the system. The remote controller can perform an objective refraction via one of two types of autonomous auto refractor methods (handheld auto refractor and/or Autonomous robotic auto refractor. Where the remote controller can control the TREES to perform a indirect to direct subjective refraction service by controlling the optometry unit table, phoropter arm, room lights, auto-refractor, vision chart softwares, and video conferencing system via the Telepresence robot.

FIG. 10 illustrates an embodiment of one of a plurality of option that can be presented to user to provide vision screening via the telepresence robot system. Including vision chart software to display vision optotypes, calibration control, Lens clock for astigmatism verification, color vision plates, and also possible vision therapy softwares. Where the controller can remote control the telepresence robot to also be able to use the speech to text software to gather examination data.

FIG. 11 illustrates an embodiment of a remote controller anywhere int the world with his electronic device and mobile application running to interconnect to the telepresence robot eye examination system. Where the remote controller can perform remote test to gather objective refractions, pupillary distance measurements, ocular health examinations, temperature checks, vision acuity and lensometry readings via the telepresence robot system and the main control base-MCB.

FIG. 12 illustrates an embodiment of a remote controller with his electronic device running mobile application to interconnect to the telepresence robot eye examination system. Where the controller can control and view the MCB and provide a realtime Vision acuity examination using video, audio, and speech to text recording systems from anywhere in the world.

FIG. 13 illustrates an embodiment of a a remote controller with his electronic device running mobile application to interconnect to the telepresence robot eye examination system. Where the controller can control and view the MCB and provide visual field examination via mobile application and/or softwares running on the virtual reality headset and/or MCB that are emulated and sent to the Virtual reality headset. Where the option of having a physical assistant helping the user is possible but not necessary as the patient can be the assistant.

FIG. 14 illustrates an embodiment of a a remote controller with his electronic device running mobile application to interconnect to the telepresence robot eye examination system. Where the controller can control and view the MCB and provide the results of the examination to the user. Where the results maybe signed eyeglasses prescription and/or a vision screening report. Where both can be printed onto the telepresence robot system and/or emailed to the user. Where in some cases and system used a transaction hash can be used to prevent prescription fraud.

FIG. 15 illustrates an embodiment of a a remote controller with his electronic device running mobile application to interconnect to the telepresence robot eye examination system. Where the controller can control and view the TREES and provide objective and subjective refraction via the telepresence robot eye examination system TREES. The following digital subjective refractor and/or digital phoropter may be controlled via the MCB and/or telepresence robot device via bluetooth, wired and/or wifi depending on the subjective refractor, digital variable lens phoropter or digital robotic phoropter used.

FIG. 16 illustrates an embodiment of a remote controller with his electronic device running mobile application to interconnect to the telepresence robot eye examination system. Where the controller can control and view the TREES and provide subjective refraction using the telepresence robot screen as a vision chart and as the proper calibrated distance. Where the user may be in any location in the world and the controller can be in any location in the world. Where in some cases the option of having the patient use a microphone to sing karaoke song lyrics to determine best corrected vision acuity as a fun and interactive option.

FIG. 17 illustrates an embodiment of a method to deliver multiple remote connections, using other remote controllers portable electronic devices as intermediary connection. Where one delegated remote controller can control the TREE, MCB and/or any other remote controller PED with a secure connection. Where one remote controller using multiple PED inter connection can perform eye examination, consultation and vision screening if needed. Where at least one of the remote controllers can be in any Geolocation 1, 2, 3, or 4.

FIG. 18 illustrates an embodiment of a remote controller with his electronic device running mobile application to interconnect to the MCB. Where the controller can control and view a facial and eye infrared thermography via realtime or near realtime of a patient (user).

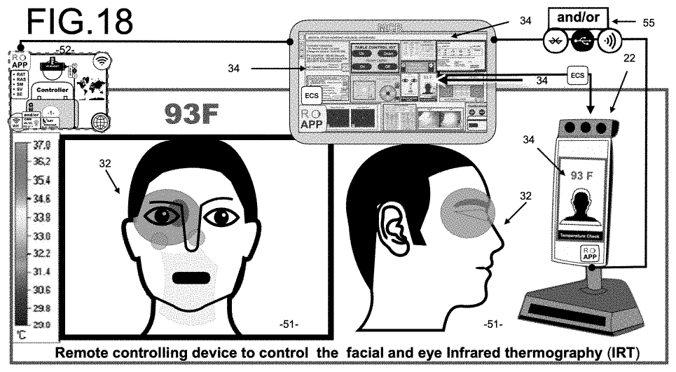

### DETAILED DESCRIPTION

The present disclosure provides a method and system for utilizing a telepresence robot eye examination system (TREES 60) to perform eye examination and vision screening to a user 32. The TREES 60 brings modularity and maneuverability for a remote controller to travel around multiple rooms and/or location to providing vision services. The TREES 60 extends the device's capabilities bringing a new approach of providing remote medical examinations from anywhere in the world. By incorporating a Telepresence robot 19 with computer system smart hubs 39 (MCB 4 15) and ophthalmic equipment (EES 2), we enhance current equipment systems by incorporating IoT devices and connection emulation software, screen mirroring, and control into telemedicine robotics.

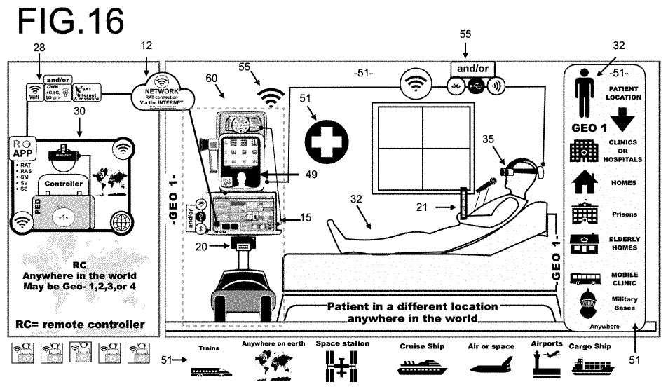

The TREES 60 will run at least one type of remote administration tool 50 technology category softwares (RAT or RAS 50), providing system access and screen sharing capabilities to an on-demand remote controller (RC 30). This provides several advantages to the currently outdated telemedicine systems. The TREES 60 allows a remote controller (RC 30 29) to connect to a second electronic device called an (MCB 4 15) that controls the control software and hardware of the ophthalmic medical equipment (EES 2). The remote controller via the TREES 60 can deliver eye examination, vision screening, and/or vision therapy 13 to a user 32 from anywhere in the world (51, 52, 53, 54). The user 32 may be a patient, customer, student, prisoner, astronaut and/or any human person on earth, and/or on earth orbit (FIG. 16; 21, 35, 51, 32).

We propose utilizing at least one type of main control base computer system (MCB 4 15). The MCB 4 15 works like as a combination computer system, smart home hub 39, emulating base and its adapters. The MCB 4 15 will run at least one operating system, has an internet connection 55, Bluetooth connection 55, remote administration tool 50 technology software 50, mobile applications 50, emulating software 63, screen view control software 63, and runs at least one other software of the RAT category. The MCB 4 15 is inter-connected (IC 55) via wired, Bluetooth and/or wifi to at least one other first electronic device (FED 58) with the operating system, and at least one equipment control software that controls medical ophthalmic equipment. The medical ophthalmic equipment coupled 55 (via wired, Bluetooth and/or wifi) to its device and control software will be called the EES 2 during this patent.

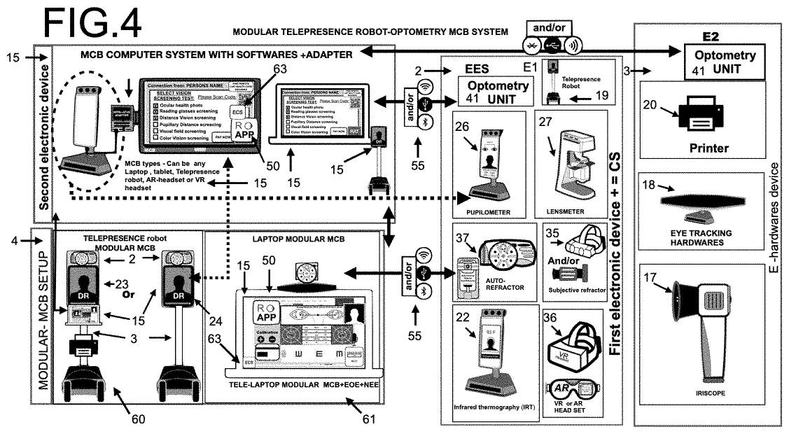

The EES 2 may be an embedded or non-embedded system and connected, coupled and/or pared to at least one type of medical, ophthalmic and/or diagnostic equipment from any maker. According to some medical equipment in the market, we may see the first electronic device coupling, connecting and/or paring to the medical ophthalmic equipment via wired, Bluetooth and/or wifi. The (MCB 4 15) is coupled and/or paired to at least one EES 2. The telepresence robot 19 may also be an EES 2 and/or telepresence equipment from any maker. The telepresence robot 19 may be a first and/or second electronic device MCB 4 15, depending on the system and configuration used (FIG. 4; 60, 61, 15, 4, 2, 3).

In certain embodiments, the first electronic device FED 58 with an operating system and equipment control software can also be called the EES 2. At least one FED 58 may be a portable electronic device embedded to the equipment but running equipment control software or mobile applications. The EES 2 may be embedded or non embedded onto the equipment via inter-connection (IC 55) coupled, connected and/or paired to connect to the medical ophthalmic, optical, robotic and/or equipment. The second electronic device (MCB 4 15) with its operating system and softwares is called the MCB 4 15. The MCB 4 15 software inter-connects to at least one EES 2 via wired, Bluetooth, and/or wifi and using emulating and control softwares 63. The connected EES 2 graphical user 32 interface is emulated onto the MCB 4 15 to provide remote control and screen view to the MCB 4 15 graphical user interface GUI.

The MCB 4 15 also provides access from a remote controller's RC 30 third electronic device PED 1 to remote access the graphical user 32 interface via at least one type of secure connection and sign-in method. This form RAT inter-connection from PED 1 and MCB 4 15 and the MCB 4 15 to EES 2 facilitates the Indirect to direct control method. The MCB 4 15 to EES 2 inter-connection to medical ophthalmic equipment and telepresence robot 19 from any maker comprises the embodiment of an indirect to direct connection. The inter-connection (IC) from the MCB 4 15 to the EES 2 emulates 63 the EES 2 software that can control the medical ophthalmic equipment and telepresence robot 19 to achieve control to the medical ophthalmic equipment remotely.

The extra equipment without electronic devices will be referred to as medical ophthalmic equipment. This involves a medical ophthalmic table stand 41 with a phoropter arm 41, iris-cope 17, and printer 20. The equipment can be controlled via wired and/or wireless connection such as: Zig-bee, Z-wave, matter, wifi and/or Bluetooth, depending on the equipment controller used and the intermediate MCB used. The EES 2 can be a medical, optical, robotic, ophthalmic, diagnosing, and/or examination equipment device from one and/or any maker model in the world market. Also, the equipment can be from any maker and model as long as it has wireless or wired capabilities to connect to the MCB 4 15.

As mentioned above, in some situations, any of the EES 2 may be an embedded or non-embedded system from any equipment maker. Some of the described first electronic devices FED 58 and equipment are via 55 wired connections, coupling, and/or Bluetooth pairing to the equipment E1 proposed in this patent are: digital pupillary distance meter DPDM 26, digital lensmeter 27, digital automatic refractor DAR, digital augmented reality headset ARH, digital virtual reality headset VRH, digital telepresence robot 19, digital variable liquid lens phoropter 35, digital optometer, digital automatic phoropter 35, variable liquid lens focometer, digital focometer and/or automatic digital phoropter 35. In some cases, the FED 58 may be from a different equipment maker but may have the equipment control software or mobile application that works to control the medical ophthalmic equipment.

The second electronic device SED 58, also called the MCB 4 15, includes at least one operating system and software comprising at least one or a combination of the following mobile application and/or software: The following may run any operating system such as Subgraph OS, Android, Microsoft OS, Windows IOT, IOS, HAOS, Ipredia OS, Discreete Linux, BlackARC 30h Linux, Kali Linux, Subgraph OS, Chrome OS, LINUX, Android, Harmony OS, Fire OS, Qubes OS, IOT-OS and/or any other operating systems open souRC 30e and/or commercial in the world market. The TREES 60 may also have a non-embedded, embedded system, kiosk software, mobile applications, open-source software, and/or commercial software from any company or maker. The following mobile applications and/or software can be executed via the MCB 4 15 and/or (TREES 60) such as speech to text (STT 57), text to speech (TTS 57), AI, ML, emulating 63, remote administration tool 50 technology software RAT, remote access RAS, mirroring, remote control, smart-home assistant 31, home automation, virtual boxes, and/or multi emulating software 63 if needed.

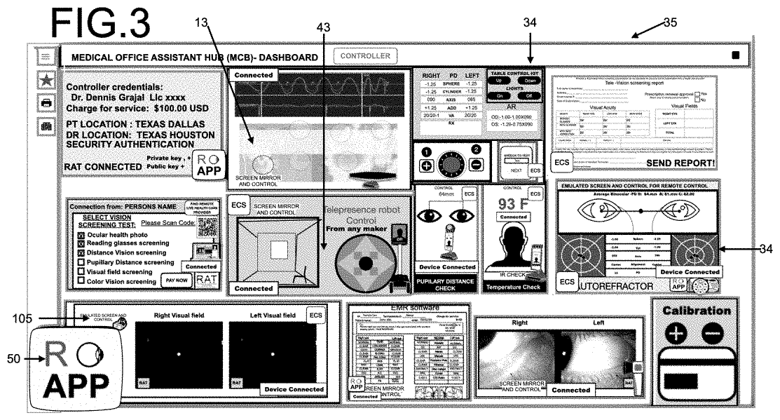

FIG. 3 illustrates a pictorial diagram of at least one form of an MCB 4 15 graphical user interface using (dashboard) setup 34. The dashboard shows the GUI 25 of control software emulated 63 onto the TREES 60-MCB 4 15 dashboard, including electronic medical records EMR, digital auto-refractor control, digital lensmeter 27, digital phoropter 35, telepresence robot 19, visual field 40, ocular cameras, digital pupillary distance meter, optometry table unit 41 control, and/or temperature check. Also, the MCB 4 15 has the ability to add to dashboard software running on the MCB 4 15 base that is not emulated, such as eye-tracking, ocular camera, kiosk software, and the payment portal with patient and provider names.

The dashboard on (FIG. 3) depicts multiple emulated 63 graphical user interfaces 24 of the control software and/or mobile application of the following: Digital automatic refractor, digital phoropter 35, digital lensmeter 27, digital VR visual field, telepresence robot 19 control, digital pupillary distance meter DPDM 26, digital Infrared temperature check, and/or any other digital subjective refractor apparatus software, where the subjective refractor may be a digital phoropter 35, digital variable lens phoropter 35, and/or digital focometer. Additional software and/or mobile applications running on the MCB 4 15 may be eye-tracking software ETS, optotype calibration 16 software; iriscope 17 software and remote control IOT optometry table unit 41 controller 42 and software 42.

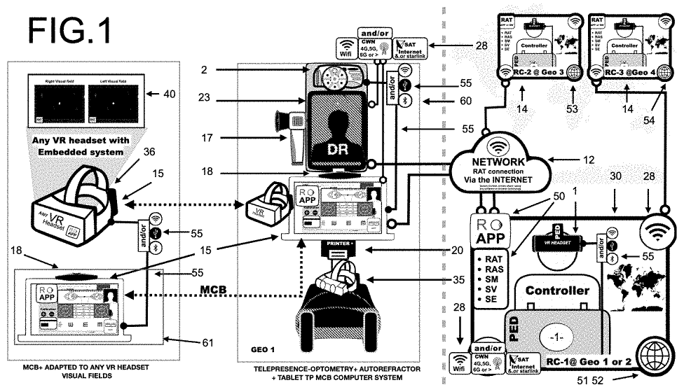

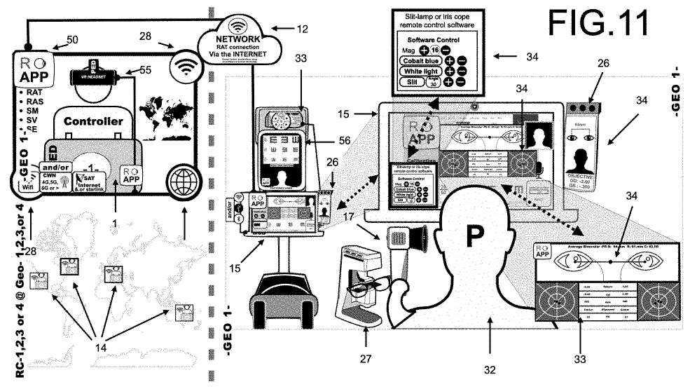

Referring to FIG. 1, FIG. 3, FIG. 4, and FIG. 11. The TREES 60 with its MCB 4 15 can have a series of software running executed onto its operating system and/or emulated onto its graphical user interface 34 via emulation remote control software 63. Some of the software or mobile application that may run on the MCB 4 15 are as follows: (EMR 11) electronic medical record software, visual field testing software 40, screen sharing, screen mirroring, Remote administration tool 50, Remote access, screen control, compatibility layer, eye tracking analysis, printer 20 drivers, ocular imaging, cryptocurrency wallets, pupillary distance meter, telepresence control, GPS MAP APP 9, audio-video communication 49, text to speech (TTS 57), speech to text (STT 57), vision chart software (VCS 56), color vision, browsers, vision therapy 13, smart control hub, calibration softwares 16, file sharing, and/or emulating 63.

The MCB 4 15 may also have emulated remote view and control 63 to the first electronic devices EES 2, thereby controlling any equipment via intermediary methods. Whereby the equipment control software runs on the first electronic devices, EES 2 include temperature check, autonomous auto-refractor 33, handheld auto-refractor AR, digital pupillary distance meter DPDM 26, digital lensmeter 27, digital phoropter 35 software, digital focometer 35, digital automatic phoropter 35 softwares, digital optometer 35, subjective refraction software 45, vision chart software 56 and/or visual field testing software 40.

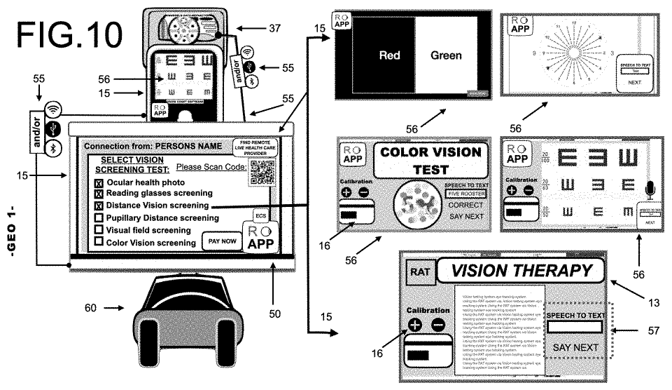

Referring to FIG. 10, the vision chart software VCS 56 and extra softwares maybe be a mobile application that may run on the telepresence device and/or the MCB 4 15 device. The softwares can be executed on any (TREES 60) devices via control by the remote controller. The vision chart software (VCS 56) or mobile application may be of any maker open source and/or non-open source. The Optotype Calibration 16 can be done via distance from the user 32, and the (TREES 60) display device is possible. The vision chart software 56 includes color plates, Optotypes, numbers, figures, characters, red-green dua-chromes, vision therapy VT softwares 13 and/or lens clock. The Lens clock will be used for astigmatism corrections and vision screening purposes.

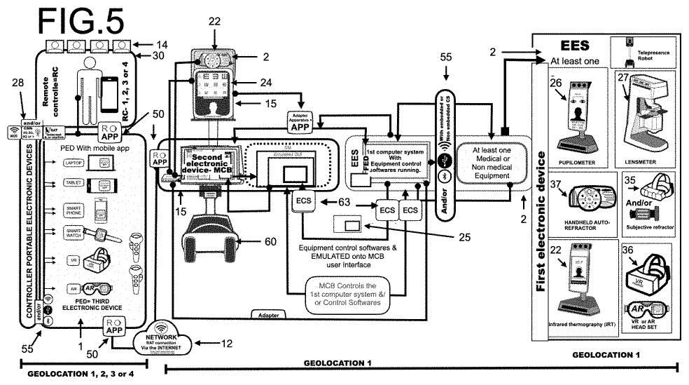

Referring to FIG. 4 and FIG. 5, In certain embodiments, the MCB 4 15 may also be coupled, connected and/or paired to at least one first, second, and/or third electronic device. The coupling, connected and/or pairing maybe via Bluetooth, wifi, and/or wired connection to provide indirect to direct control from at least one other controller's third computer device PED 1 controllers device to at least one second and/or first electronic devices with its equipment control software for hardware, robotic, medical, ophthalmic and/or diagnosing equipment. The second electronic device used as a main control base MCB 4 15 presented herein may be implemented using various types of electronic devices or combinations of devices. Examples of such electronic devices include smart hubs 39, smartphones, user 32 equipment, laptop computers, tails (on usb sticks), portable computers, slate PCs, tablets, ultrabooks, smartwatches, smart glasses, virtual reality headsets displays, and/or augmented reality head-mounted displays; whereby any of the electronic devices terminal types may be of any operating system, maker and models.

As mentioned above, the MCB 4 15 may also have cellular wireless networks, wifi, ethernet, and/or cellular satellite network 28 capabilities depending on the system used and/or adapted equipment coupled, connected, and/or paired 55 to the MCB 4 15. The main control base system may be constructed to have at least one or a combination of the following operating systems: Windows, Harmony OS, open-source IOT-OS, Apple OS, macOS, Linux, Android, UNIX, chrome os, Apple's iOS, Internet operating systems, embedded system, embedded system or any operating systems and/or combination described thereof. In some cases, the main control base MCB 4 15 may only have at least one operating system depending on the system constructed and tasks.

The second electronic device and/or MCB 59 15 4 may have one of a combination of software that enables it to work with multifunction capabilities MCB. Furthermore in some situations it may run additional softwares via virtual boxes VB, virtual machine VM, emulators GUI, emulating control, mirroring, remote administration tool 50 technology, kiosk systems, smart hub 39, virtualization, and/or compatibility layer software (FIG. 4; 63,15, 2,3).

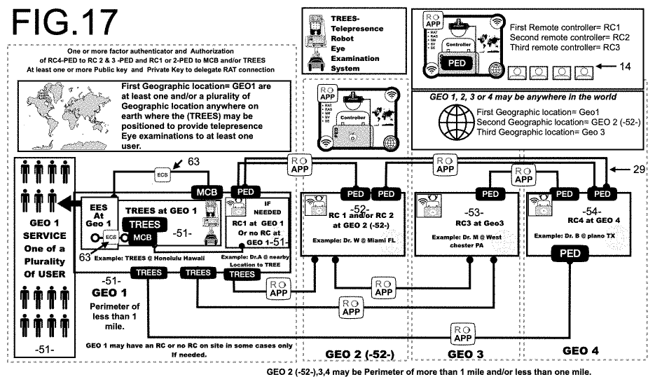

Referring to FIG. 4 and FIG. 5, the main control base MCB 4 15 may be of any type of device such as one but can also be one or a combination of laptop, tablet, virtual reality headset, telepresence robot device 24 23, and/or augmented reality headset with software from any maker or model. The third computer device PED 1 may be of any type of device such as one or a combination of laptop, tablet, virtual reality headset, and/or augmented reality headset with software. The TREES 60 is modular and is capable of being adapted for telemedicine and telepresence robotic evaluation via an MCB 4 15 with remote administration tool 50 technology and/or remote access software 50. The connection from a remote controller RC 30 with a PED 1 running mobile application to perform a telepresence robot 19 eye examination and/or vision screening to a user 32 from anywhere in the world (FIG. 17).

Referring to FIG. 5, The telepresence robot 19 is adapted to an MCB 4 15. The (TREES 60) is a modular combination system. The telepresence robot's 19 main control base is connected to at least one EES 2. The EES 2 functions to control at least one E1 piece of equipment. At the same time, we were using at least one software to interconnect medical, robotic, non-medical, or ophthalmic equipment via a modular setup system. The system can be used to provide telemedicine vision screening and examination service via remote administration tool 50s, screen share, and screen view technology. In certain embodiments, the main control base computer system may function partially as a smart hub using at least one software and can be used as an intermediary smart hub controller to control and emulate 63 via (mirroring, emulation, remote access and/or screen control) to at least one health monitoring equipment, eye-tracking hardware 18, medical equipment, ophthalmic equipment, telemedicine laptop kiosk 61, telepresence robot 19, and/or instruments.

Where at least one instrument or equipment may be coupled to at least one main control base MCB 4 15 and can be controlled by a remote controller 30 using remote administration tool 50, remote access tool 50, screen share and/or screen control RAT 50 technology to aid in the telemedicine, and tele robotic eye examination connection. Where the remote controller RC 30 can be any health care provider and/or assistant 31 to provide online on demand vision screening services via the intermediary computer system called the MCB 4 15 and the telepresence robot 19. In some cases a physical assistant 31 may be at the location to help the user 32 with the eye examination and/or vision screenings. The assistant 31 can be any human being capable to help in the same location where the user 32 is located (FIG. 16; 51).

Referring to FIG. 16, a virtual, physical and/or remote assistant 31 may also aid in the eye examination and/or any vision screening needed to be performed to the at lease one user 32 in geolocation 1 (GEO1) 51. The location 51 of the the user 32 maybe anywhere in the world in a room, outdoor, indoor, kiosk, prison, hospital, vehicle, airplane, oil rig, trailer, mobile trailer, outer spaces station, building, military base installation, cruise ship, ship, elderly home, clinic, hospital, optical shop, and/or anywhere in the world (FIG. 16; 51). In some cases the user 32 and/or an assistant 31 may request a vision examination via mobile application and/or browser using a booking company, or booking website. The controller will receive GPS and instruction of the location and place needed for service in the world.

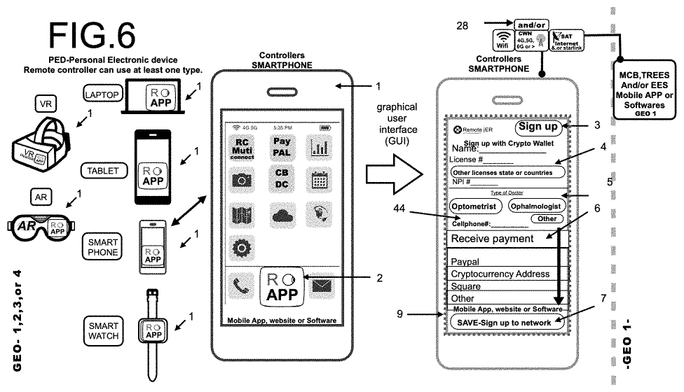

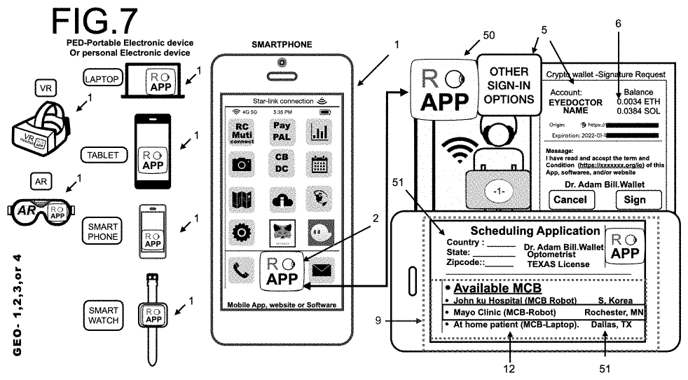

The information of the location 51 52 53 54 and time and space (GEO1 51) of the service needed can also be provided by secondary system, network of controller, tele-health, or any form of medical appointment booking platforms. Payment for service rendered by be via medical, vision, and/or out of pocket payment. Payment options for the user 32 to the remote controller RC 30 or network of remote controller RC 30 via the for (TREES 60) may be via any of the following depending on the payment system used 6: electronic cash, any central bank digital currency, stable coins, cryptocurrency, cryptocurrency tokens, insurance digital token systems, credit card, PayPal, WeChat pay, Tencent pay, membership payment system, Alipay, QQ tokens, eye exam service tokens, and/or any form of distributed ledger payment gateway. (FIG. 6; 44,3,4,5,6,7, 1) (FIG. 7; 50,5,6,51,9,12, 51, 2, 1).

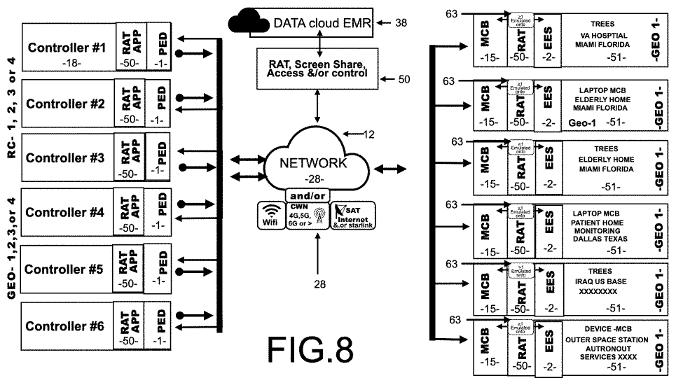

According to an embodiment of the present invention, an (EES 2) is a (medical, optical, robotic, and/or ophthalmic equipment coupled, connected and/or paired to at least one first electronic device FED 58) (FIG. 8, FIG. 5). The first electronic device (FED 58) may run operating system, and may run an equipment control softwares or mobile application to control the equipment from any maker or model. We decided to separate the both parts in the embodiment to show that the connection is achieved via any controlling module and that is coupled to any types of medical, ophthalmic, and/or robotic equipment. Where the method to enhance any medical, robotic and/or ophthalmic equipment in the market to have remote control capabilities is achieved via at connection from the (FED 58) EES 2 and the MCB 4 15 if needed.

As mentioned above, the first electronic device (FED 58) may also have cellular wireless networks (LTE, 4G, 5G, 6G and/or greater), wifi, ethernet, and/or satellite cellular network 28 capabilities depending on the first electronic device used. The equipment is coupled, connected and/or paired 55 to the FED 58 and run mobile application and/or software to become an equipment controller. Some manufacturer may have one of many types of FED 58 may be constructed to have at least one or combination of the following operating systems: Windows, Harmony OS, open souRC 30e IOT-OS, Apple OS, macOS, Linux, Android, UNIX, chrome os, Apple's iOS and/or any operating system. The operating systems could be also an embedded system, non embedded system operating systems and/or combination described thereof. In some cases the first electronic device FED 58 and/or with OS and softwares connected to equipment EES 2 may only have at lease one operating system depending on the system constructed by its maker and model.

The first electronic device FED 58 may be made by other companies not associated with the equipment company but use one of a combination of softwares or mobile applications that enables it to work as an equipment controller via equipment control softwares and/or mobile applications. In some cases the first electronic device FED 58 may be a computer system device such as personal computer in most older systems but it can also be portable electronic devices such as tablet, smartphone, virtual reality headsets 36, augmented reality headsets 36, and/or laptops. Other examples of such first electronic device FED 58 include smart phones, user 32 equipment, laptop computers, portable computers, slate PCs, tablet, ultra books, smart watches, smart glasses, virtual reality head sets displays, and/or augmented reality head mounted displays; whereby any of the electronic devices terminal types may be of any operating system, maker and models.

In some cases a RAT 50 direct to direct connection from the controller third electronic device PED 1 and at least one first electronic device EES 2 can be achieved if needed and in some equipment in this embodiment the telepresence robot 19 to form the options for a TREES 60. Currently many medical and/or ophthalmic equipment in the market have personal computers, and/or electronic devices coupled, connected and/or pared to equipment but lack remote control functionality. Most EES 2 are only configured to control robotic, medical, and/or ophthalmic equipment via control softwares running on an operating system with no other options. Unfortunately some older and new medical equipment in the market were not build to have internet connectivity and are were never able to be enhancing to have IOT (internet of thing capabilities).

Although some first electronic devices FED 58 have some internet capabilities and/or wireless capabilities like bluetooth and/or wifi, some of these FED 58 running equipment control softwares may be easily be enhance to have remote control capabilities via our proposed (RAT 50 direct to direct and/or RAT 50 indirect to direct method). From our previous published, research and development we have achieved abilities to enhance any medical, or ophthalmic system, to have capabilities of being remote controlled via using wifi, bluetooth 55, zig-bee 55, z-wave 55, and/or cellular wireless network 55 using mobile application and/or softwares. After further testing we managed to use an MCB 4 15, EES 2, emulating softwares 63 and RAT softwares 50 to control any equipment in the market. At least one of a plurality of first electronic device EES 2 may be coupled and/or paired 55 with a second electronic device MCB 4 15 and the second electronic device MCB 4 15 may be coupled, connected and/or paired to a third electronic device PED 1 to achieve remote control. The pairing connection can be performed via any communication method described.

Furthermore, the inter-connection 55 of EES 2 and MCB 4 15 according to the communication method may be executed using at least one of the communication method technologies (CMT): cellular wireless networks, satellite internet network 12, (Starlink), Bluetooth, ZigBee, NFC (Near Field Communication), infrared, remote administration tool 50 technology 50, remote access software technology 50, Wi-Fi and/or Wi-Fi Direct depending on at least one equipment and/or any combination configurations of described above. The emulated connection 63 may be made in a wired manner such as HDMI (High Definition Multimedia Interface), usb connection and/or any other type of connection depending on the equipment and/or equipment maker used. The connection, however, may directly emulate graphical user interface 34 of the EES 2 to the display device of the second electronic device MCB with or without an internet access point through a Wi-Fi depending on the system and/or configuration used.

A remote controllers RC 30 or network of remote controllers 14 connection from the MCB 4 15 to the EES 2 graphical user interface can be achieved also via the remote administration tool 50 RAT, emulating softwares 63 and/or control softwares 63 used. In addition, for example, the remote emulating control and viewing connection may be performed between the equipment control softwares running on the first electronic device EES 2 and to the second electronic device MCB 4 15. The MCB 4 15 may be inter-connected via a secure connection to a controllers third electronic device PED 1 graphical user interface via a Wi-Fi, bluetooth, cellular wireless networks and/or satellite internet networks 12 depending on the wireless network 12 28 used and device used. The inter-connection providing the controller via a mobile application and/or software to perform eye examinations via the TREES 60 to at least one user 32 in the same (GEO1 51), remote and/or geographically distanced location (GEO2 52, GEO3 53, or GEO4 54). The method of configuration of the system of the invention involves at least one of a plurality of first electronic devices FED 58 running equipment control software EES 2 coupled (IC 55) to its medical, ophthalmic, optical and/or robotic equipment hardware to function as a multi equipment controller.

Furthermore a method for emulating 63 at least one of a plurality of EES 2 graphical user interfaces with its equipment control softwares GUI emulated onto a secondary electronic device MCB 4 15 graphical user interface and a method to control at least one of the first computer device FED 58 EES 2 GUI user interface via at least one MCB 4 15 with GUI user interface emulated and onto at least one third computer device PED 1. Where emulated 63 GUI 34 is viewed and controllable by human (remote controller) via at least one third computer device PED 1 running mobile application and/or softwares 50. The inter-connection 55 to the MCB 4 &/or TREES 15 to provide realtime and/or near realtime (TREE) telepresence robot 19 eye examination and/or vision screening to any user 32 in and from anywhere in the world 51 (on land, water, air, space orbit and/or outer space 51).

Where according to the present invention, at least one remote controller 30 connection request is received from a controllers third electronic device PED 1 mobile application APP and/or softwares or multi RAT method 50 29. The first electronic device EES 2 device coupled to any equipment or robot, a second electronic device MCB 4 15 device connected, paired and/or coupled 55 to at least one first electronic device that may be connected, paired and/or coupled 55 to an equipment. The remote controllers third electronic device (PED 1) connected at least one MCB 4 15 that controls at least one first electronic device EES 2 that control at least one equipment and/or robot. Where multi emulated graphical user 32 interface screens of the first electronic device and second electronic devices MCB and with its emulated softwares 63 of at least one of a first and second external device can be displayed and is able to be controlled via the remote controller 30 PED 1 29 50 from anywhere in the world 51 52 53 54. The remote controller 30 PED 1 running mobile application 50 used to perform a health check, vision therapy, eye examination, vision screening, objective refractions and/or subjective refractions to a user 32 in the same, remote and/or distanced location 51 52 53 54.

Whereby at least one remote controller 30 with at least one electronic device called the PED 1 will view and control the (TREES 60 and/or MCB 4 15) or other remote controllers PED 1. The remote controller uses at least one mobile application from anywhere in the world can control the TREES 60 to perform a realtime or near realtime: patient positioning, vision screening, visual fields, eye tracking examinations, vision therapy 13, objective refractions 37 33, subjective refractions, temperature monitoring, pupillary distance measurements, color vision examinations, eyeglasses prescription 47 evaluations, prescription 47 renewals, vision screening reports, and/or external ocular examinations using remote administration tool 50 softwares and an internet connection (FIG. 11).

Referring to FIG. 17 and FIG. 8, the first remote controller with PED 1 and mobile application running can interconnect to the TREES 60 and/or MCB 4 15 to control the medical ophthalmic equipment and equipment. The first remote controller can controller the TREES 60 and/or MCB 4 15 or other remote controller PED 1. The TREES 60 is located in the first geographic location 51, abbreviated as GEO1 51. The (GEO1 51) comprises one of a plurality of locations a TREES 60 and/or MCB 4 15 is located. The first remote controller can be in the same geographic location GEO1 51 as the TREES 60 and user 32 or off-site from on a second geographic location (GEO2 52). The TREES 60 may be at a distance less than 1 mile from the user 32. However, the TREES 60 may be maneuver remotely to at least less than 1 foot from the user 32 within GEO1 51.

The (TREES 60, MCB 4 15, and the user 32) may also be less than a one-mile apart in the distance. A distance greater than one mile from the geographic location of GEO1 51 will be called GEO2 52. The GEO2 52 can be anywhere in the world. The first remote controller 30 is connected to at least one PED 1 with a mobile application to control the TREES 60 and MCB 4 15. A second remote controller can also be in GEO2 52 also and may connect 50 to at least one TREES 60 or first remote controller PED 1 to control the TREES 60 or MCB 4 15. A third remote controller with a PED 1 50 may connect 50 to the second remote controller PED 1, First remote controller PED 1, TREES 60, and MCB 4 15.

A fourth remote controller RC 30 may be located anywhere in the world in (GEO4 54) and can connect 50 to the first remote controller RC 30 PED 1, second remote controller PED 1, Third remote controller PED 1, TREES 60 and/or MCB 4 15. Although in most cases, we will use one or two remote controllers 30. We have used a method of delegation of service via multi-screen control and screen sharing using multiple remote administration tool 50 software RAT 50 and Remote access software RAS 50. At least two remote controllers with PED 1 can screen sharing, screen control, and screen viewing via a secure inter-connection between multiple PED 1, TREES 60, and MCB 4 15. The inter-connection aids in providing multi-location remote administration tool 50 RAT eye examinations, vision screening, verification via multi networking, and remote access tool 50 RAT connections MLRC 30 29.

Such instrumentation that can be adapted to the TREES 60 may includes devices with or without their embedded computers system and control softwares. Example are of instrumentation and equipment can be: Iris-cope 17, telepresence robots 19, virtual reality headset 36, augmented reality headset 36, pupil-meter 26, lens meter 27, eye tracking device 18, Infrared thermographic apparatus 22, vision chart softwares VCS 56, color vision softwares 56, digital optometer 35, digital phoropter 35, digital focometer 35, robotic optometry table, robotic and phoropter arm unit 41 and/or auto-refractor 37 among others (FIG. 4).

Referring to FIG. 1, illustrates an embodiment of a telepresence robot 19 eye examination system TREES 60 from any maker with its multiple first computer device with control softwares and equipment EES 2 connected to a first computer device called the MCB 4 15. Where at least one remote controller with a virtual reality headset and a laptop computer is inter-connected 50 28 via the internet network 12 28 and using at least one softwares and mobile application 50 to achieve remote administration tool 50, remote access tool 50, screen mirroring, screen viewing and/or screen emulating control to internet connect with at least one MCB 4 15 from anywhere in the world (51,52, 53, 54). The telepresence robot 19 device and is assembled to have multiple equipments EES 2 adapted to it housing. The EES 2 being any handheld or autonomous auto refractor 37, iris-scope 17, virtual realty headset, subjective refractor, printer 20, and digital pupillary distance meter 26. Where virtual reality headset 36 of any maker may be running a mobile application that can be connected to the MCB 4 15 thereby being remotely viewed and controlled by at least one remote controller 30.

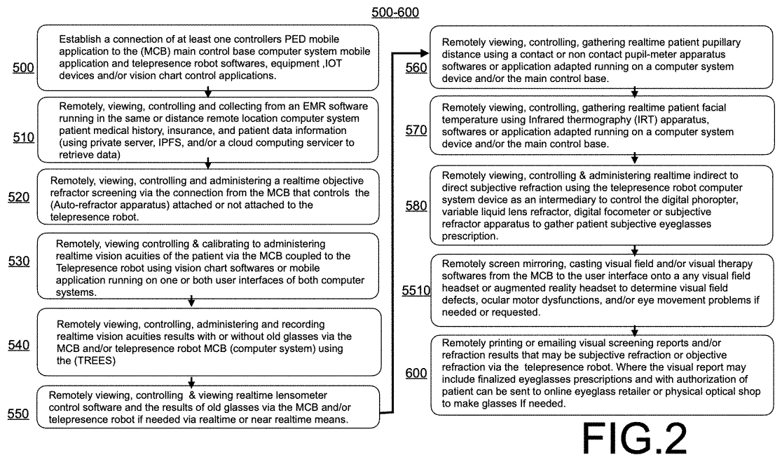

On FIG. 2 we show a method of providing telepresence eye examinations, whereby the remote controller RC 30 may establish connection 50 12 15 55 with the MCB 4 15 and telepresence robot 19 setup. The controller will navigate the telepresence robot 19 to the location needed for services 51. Where the remote controller 30 can then remotely, navigate, control and collect the patients data from the EMR 11 38, (Cloud and/or IPFS) 38 and establish a video audio, and/or text connection 49 with the user 32. The EMR 11 may be running on the same or distance computer device on any other geolocation (51, 53, 54).

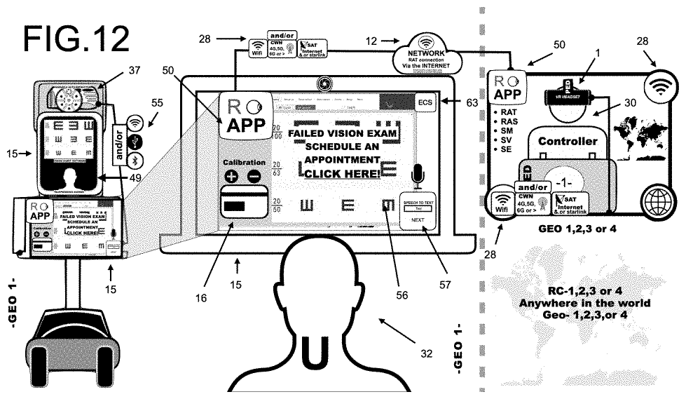

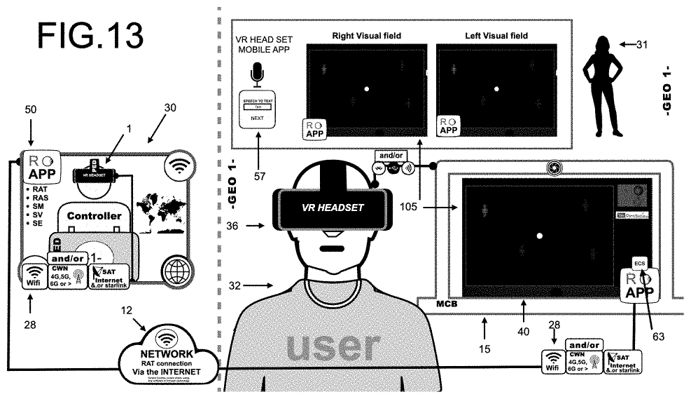

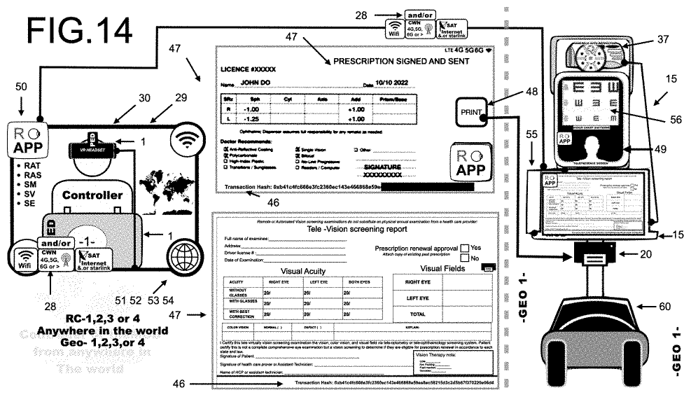

At least one remote controller 30 can begin administering a realtime connection to perform an objective refraction, vision screening, color vision, subjective refraction. The controller can use camera device such as an iris-scope 17 and/or ocular camera 17 to view the patients external and/or internal eye structures (FIG. 3). The remote controller 30 can then send out or print out 48, email the finalized eyeglasses prescription, and/or vision screening report 47 to the user 32 (patient) providing any of the following (FIG. 14): vision screening report 47, eyeglasses Prescription 47, and/or referral 47 to another health care provider at any other geolocation (FIG. 17; 30,51,53,54). The remote controller 30 has the option of performing a visual field to user using an augmented reality 36 and/or virtual reality headset 36 from any maker or model. (FIG. 12)(FIG. 13), (FIG. 14).

Referring to the drawings more particularly by reference numbers, FIG. 4 shows different forms of computer systems that can become an MCB 4 15 main control base. Where a main control base 15 can be a laptop, tablet, telepresence robot, computer, virtual reality headset, and/or augmented reality headset. Where the MCB 4 15 can be setup configured to connect to at least one EES 2 that may be connected to an examination equipment (E1) 2 3. The equipment (E1) 2 may be a medical device 2 3, ophthalmic equipment 2 3, telepresence robot 19, and/or optical examination equipment. The telepresence robot 19, MCB 4 15 and EES 2 modular system called the (TREES 60) telepresence eye exam system.

Where this MCB 4 15 modular system can be used to provide a telemedicine connection to a patient via at least one remote controller 30 portable electronic device 1 using at least one computer softwares 50. The main control base 15 setup system includes softwares that enable at least one of a series of EES 2 and equipments 2 3 to connect to the main control base station and to connect to a remote controllers 30 third electronic device PED 1 with mobile application 50. The Main control base 15 station may be coupled, connected and/or paired 55 to the instruments or equipment controllers. The (third electronic device also called the PED 1) may be coupled, connected and/or pared 50 12 to the main control base 15 via internet network 28 12 using at least one mobile application 50 and at least one software 50 (FIG. 1).

By way of example, the coupled, connection, and/or pairing 55 maybe of via (WI-FI, wired, bluetooth, cellphone wireless networks and/or wired cable connection) 55 or combination of any of the types mentioned thereof and depending on the system built and equipment used. The MCB 4 15 may be coupled to ophthalmic equipment electronic device and/or embedded system computing device to achieve an indirect to direct emulating view control method 63. By way of example, the main control base 15 may be connected to a wireless router 28 if needed. Alternatively, the telepresence robot 19 may have a direct to direct connection and/or indirect to direct connection whereby the telepresence robot 19 can be controlled via the MCB 4 15 and/or the telepresence robot 19 computer devices.

Whereby at least one remote controller RC 30 with third electronic device PED 1 can achieve one of two types of connection to control the telepresence robot 19, A connection to the telepresence robot 19 computing device, and/or a connection to the MCB. Also via a connection of a multi emulated control system 63 on the main control base 15 via internet connection using wifi, cellular wireless networks, and/or satellite internet connection networks 28. Other methods of connecting are that a remote controller multiple different geolocation can interconnect to other remote controllers PED to achieve alternative connection to control the TREES and MCB to control the medical ophthalmic equipment and perform examination via remote control multi RAT connections (FIG. 17).

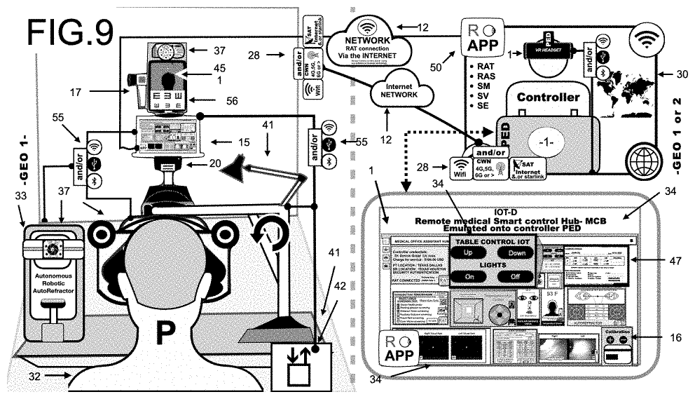

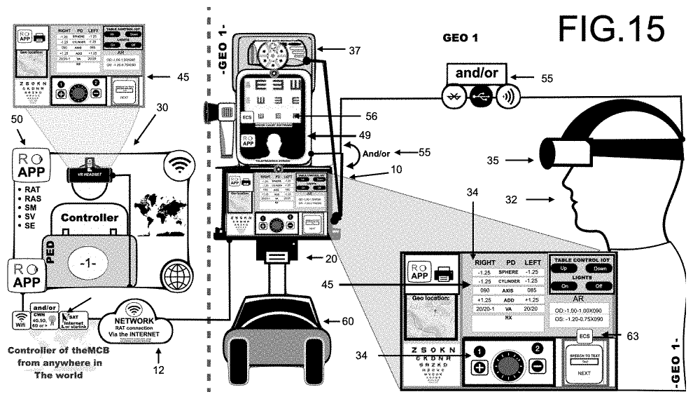

Referring to the drawings more particularly in (FIG. 15, and FIG. 16) the telepresence robot 19 is adapted to an MCB 4 15, handheld auto-refractor 37, running vision chart softwares and subjective refractor control softwares 45 via the MCB 4 15 or the telepresence computer system. Where the subjective refractor 35 can be controlled via indirect to direct means by a geographical distant 51,52, 53, 54 remote controller 30 to provide subjective refraction to a patient in a remote location. The telemedicine controller can control movement of the telemedicine robot from his personal electronic device mobile application or browser.

Where the controller can also control the movement of the telemedicine robot via the MCB 4 15 and an eye examination smart hub setup. Where all medical equipment and control software are seen via dashboard via realtime or near realtime (FIG. 3). The eye exam telepresence robot 19 includes equipment adapted such as handheld auto refractor 37, robotic autonomous auto-refractor 37, vision chart softwares, camera system, audio system, and/or subjective refractor control softwares. Where the handheld auto refractor is adapted to the telepresence robot 19 and the main control base computer system (FIG. 9).

Whereby a remote controller 30 can remotely control the auto refractor 37 to determine the patient tentative Prescription 47 from any geographic location (51,52,53,54). Then the remote controller can perform a remote controlled a subjective refraction to a user 32 using a telepresence robot and/or MCB 19 as intermediary. Where the subjective refractor softwares 45 is designated to control at least one digital phoropter 35, variable lens phoropter 35, digital optometer 35 and/or digital focometer 35 from wired or wireless connection from the telepresence computer system and/or MCB 4 15 to the subjective refractor control parts and lenses and/or base curve or liquid power lenses. The camera system that can capture and transmit to the remote controller personal electronic device via mobile application or softwares via a remote administration tool 50 technology, screen share, and screen control.

Any of the remote controller can be a health care provider such as an optometrist, ophthalmologist, nurse, and/or an assistant 31. The remote controller 30 can also be a designated artificial intelligent computing device, group of human beings and/or network of any type of controllers with other types computing device to perform eye examination and/or visions screenings. The providing medical service to at least one patient in a remote location. A human remote controller 30 personal electronic device 1 may be at least one or a combination of: a laptop, tablet, smartwatch, smartphone, virtual reality headset, and/or augmented reality headset. The remote controller may also use his personal electronic device running remote administration softwares to secure a connection to perform eye examination and/or vision screening to a user 32 anywhere in the world (51,52, 53, 54).

Referring to FIG. 6, FIG. 7 and FIG. 5, any of the remote controllers third electronic device may be any portable electronic device 1 using at least one mobile applications and/or softwares 50 to works as a remote administration tool 50 technology system. The controllers portable electronic device may be one or a combination of laptop, tablet, smartphone, smartwatch, virtual reality headset, and/or augmented reality headset. The remote controller 30 portable electronic device 1 may have at least one computer processor, memory, operating system, display screen, touch screen, a camera, antenna, keyboard, a microphone, battery, a speaker and/or any other parts components, hardwares and/or softwares that are in portable electronic devices but may be lacking one or more of the above mention depending on the portable electronic device used. A following example of possible remote controllers third electronic devices may be: tablet, smart phone, smartwatch, personal computers, laptops, notebooks, virtual reality headset, augmented reality headset, and/or combination from any maker or model in the market.

The first electronic device may be also any type portable electronic device depending on the equipment maker and system used. The first electronic device may also have at least one mobile applications and/or softwares to works as a remote administration tool 50 technology system in some cases depending on the system and connection method used. The first electronic device FED 58 may also be be a laptop, tablet, smartphone, smartwatch, virtual reality headset, and/or augmented reality headset. In some cases the first electronic device FED 58 may have mobile application running specific diagnosing and/or vision screening softwares.

Where these electronic devices may also be connected to an MCB 4 15 to provide remote connection, via a direct connection to the first electronic device, via connection from the telepresence robot 19 device, second, and/or third electronic device. The portable electronic device PED 1 may also contain an input/output device such as a virtual reality hand held controllers, computer mouse and/or keyboard. The remote controller RC 30 portable electronic device PED 1 may be located in any location In the world that is remote or distance from the main control bases MCB 4 15 and/or telepresence robot 19 TREES 60 and computer systems.

Although only one remote controller 30 can control the system at a time via encryption verification using sign in and/or cryptocurrency wallet signature request as shown (FIG. 9) (FIG. 8), the system may include a the ability for a plurality of remote controllers 30 to provide service to at least one main control base anywhere in the world via delegation platform. In general any number of telepresence robot 19 eye examination systems setups configurations may be controlled by any number of remote controller via any number of portable electronic devices depending on secure connection, needs and service needed. For example, one remote controller may be connected to a at lease of one main control base and/or telepresence robot 19 eye examination robot to delegate a connection to a second controller to perform, control and view at least one data pertaining to the user 32 receiving service.

The EES 2 the combination of first electronic device with the medical, optometry and ophthalmology equipments in the market. Additional equipment with that needs to be adapted to a computer system will also be printer 20, eye tracking hardwares and/or iris-scope 17 and will be called (E2 3). The E2 3 can also be coupled, connected and/or paired to the main control base MCB 4 15 and/or TREES 60. At least one remote controller RC 30 can remotely view and control the iriscope 17 cameras, eye tracking and eye movement diagnosis, and remotely printing visions screening reports via the TREES 60. The remote controller may use the (TREES 60 and/or MCB 4 15) to deliver user 32 with eyeglasses Prescription 47, contact lenses prescription 47, visions screening reports and/or medical referral 47s notes to user 32 anywhere in the world. In some embodiment one may use a transaction hash and time stamps on eyeglasses, contact lenses and/or vision screening reports.

Each telepresence robot 19 is attached to a main control base and its EES 2 and/or additional Equipments. Additional equipment maybe a printer 20, eye tracking hardware 18, and/or iriscope 17. Also attached to the TREES 60 housing is a cameras and, a microphones and a speakers. The microphone and speakers may be used to gather voice to text data. Furthermore, it also provides means for video audio connection 49 from the remote controller 30 to the user 32. In some setups a TREES 60 may also have an GPS, Lidar and/or additional music playing capabilities. Music playing capabilities may be karaoke music softwares depending on the softwares and mobile application used.

The following karaoke music softwares will display lyrics of an unknown song to the user 32, where the optotypes and words gradually become smaller or larger. This to determine optical vision acuity of a song to gather data via speech to text the user 32 can see during the final telemedicine vision examination. The system will also allows a user 32 to karaoke sing word via microphone input device via the TREES 60 display screens. The (TREES 60) cameras and are coupled to the remote speech to text monitor so that a controller at the remote location can can view and record the a user 32 and gather vision data. Likewise, the TREES 60 can be used so that the patient can ask the controller to have the session recorded and/or sent to his email by the controller via mp4.

After the session is over the controller may explain any finding to the user 32. The findings interring eye health and vision results. The may print or email the patient (user 32) the report that may include new eyeglasses prescription 47, vision screening report and/or referral 47 to the health care provider. The controller may also provide optional karaoke singing of the results and/or eyeglasses Prescription 47 to the patient if they would like to to make the the telepresence robot 19 eye examination more pleasant, unique and comforting.

The camera, microphones and speakers and, allow for video, audible, and/or text to speech 57 communication between the patient (user 32) and the controller 30 and/or assistant 31. The physical assistant 31 can be any human, caregiver or the user. A remote controller RC 30 can be assistant 31 and/or is health care provider such as optometrist, ophthalmologist, pediatrician and/or any medical doctor who can remotely control the TREES 60 and also access medical files contained in the TREES 60 memory. In some cases with permission from a remote controller a family remotely access the TREES 60 to view user 32 data and control the robot if perceived safe by at least one controller 30.

What is claimed is:

1. A system for providing remote administration tool controlled eye health examination and vision examination to a patient using a telepresence robot with a main control base computing device interface the system comprising:
a first geographic location, including at least one or more of: a telepresence robot, electronic device, medical device controller interface, non-medical device controllers interface, MCB, medical devices, and non-medical devices; and
wherein the telepresence robot, includes coupling, to a second electronic device and main control base (MCB); and
wherein the second electronic device MCB, including coupling at least one or more first electronic devices; and
wherein the one or more first electronic devices, include at least one medical device and non-medical devices control software's configured to be controlled to operate the devices from one or more location; and
wherein at least one first electronic device, is configured to be controlled and emulated onto the second electronic device MCB interface; and
wherein the combination of at least one Telepresence robot coupled to MCB comprises a Telepresence robot eye examination system (TREES);
wherein the TREES, is coupled to at least one medical device and non-medical device; and
wherein the TREES includes one or more electronic devices configured to control one or more medical device controller interfaces and non-medical device controllers' interfaces configured to be controlled from a human form multiple location; and
wherein the Main control base second computing device comprises:
an operating system;
a user interface;
an adapter;
a computer user interface emulator;
a Remote administration tool (RAT); and
wherein the one or more electronic devices, including the one or more user interfaces and controllers, are controllable by an MCB computing device; and
wherein the one or more electronic devices includes at least: one or more medical devices controller, IoT devices controllers, and a telepresence controller interfaces, configured to receive instruction for operating the one or more devices from one or more locations and administer one or more vision and eye health examinations for an patient via a telepresence robot session connection; and
one or more Portable Electronic Device (PED) associated with at least one human remote controller; and
wherein the telepresence robot and MCB are configured to:
establish a secure internet connection with the portable electronic device PED associated with a remote controller via a RAT connection; and
wherein the remote controller's PED is configured to work on at least one of: a Remote Administration Tool (RAT) and a Remote Access Software (RAS); and
wherein the remote controller PED comprises at least one: virtual reality headset, smartwatch, a tablet computer, a laptop computer, and a smartphone; and
wherein the remote controller's PED interface, includes at least one software configured to achieve connection to the one or more medical devices and one or more non-medical devices; and
wherein the TREES is configured to control a digital phoropter, vision chart, and one or more devices in the exam locations; and
wherein the exam location comprises a first geographic location at least one of: a mobile clinic and a non-mobile location; and
where mobile location, comprises at least one: a train, space station, cruise ship, airplane, trailer, and any vehicle; and
where non-mobile location, comprises at least one: optical shops, hospitals, homes, prisons, elderly homes, military bases, and airport's location; and
wherein the mean of controlling the TREES from a remote controllers' PED comprises an internet connection and a RAT interconnection control from any geographic location in the planet over one or more networks.

2. The system of claim 1, wherein at least one remote controller is a a health care provider and can be located anywhere in the world; and wherein the remote controller controls at least one portable electronic device (PED); and wherein the PED includes at least one mobile application, software's with remote administration tool technology and an internet network connection to control the eye examination telepresence robot.

3. The system of claim 1, wherein at least one patient may request a telepresence robot eye examination and vision screening via scheduling system.

4. The system of claim 1, wherein at least one third electronic device (PED) associated with the health care provider comprises at least one of: laptop, tablet computer, smartwatch, virtual reality headset, smartphone, PC, and augmented reality headset; and wherein the remote controller PED are can used to perform a remote vision examination and eye health examination to an patient via the telepresence robot system.

5. The system of claim 1, wherein at least one mobile application and a software are executed on at least two of any of the electronic device and computer devices; and wherein at least one computing device software may be at least one: remote administration tool RAT, emulation view and medical device control software; and wherein the medical device control software's are screen viewable, screen controllable, and remote controllable; and wherein the medical device control software's may comprise at least one of: ophthalmic equipment control software's, calibration software, distance tracking software, eye tracking software's and vision acuity software.

6. The system of claim 1, wherein the Telepresence computing device system comprises at least one of: a display screen, camera, microphone, speaker, a computer processor, and a memory, computer processor; and wherein at least one computing device is configured to receive at least one control connection from a remote controller from multiple location.

7. The system of claim 1, wherein at least one eye examination and vision screening performed by at least one remote controller, comprises control of one or more electronic devices and one or more medical device computing controller from the same, distance, remote and/or different geographic location; and wherein an remote control eye examination comprises at least one of: medical vision screenings, eye health examination, eye movement screenings, vision therapy, vision acuity screening, pupillary distance measurement, facial temperature checks, lens-meter readings, visual field testing, objective refraction and a subjective refractions; and wherein subjective refraction comprises releasing at least a finalized eye glasses prescription for service rendered to the patient.

8. The system of claim 1, wherein at least one telepresence robot is used to perform at least a vision examination and eye examination to a patient.

9. The system of claim 1, wherein at least one telepresence robot comprises at least one first electronic device with equipment control software's capable of being control from one or more location; and wherein the telepresence robot electronic device is configured to be coupled, paired and/or connected via wire, Bluetooth, infrared and/or WIFI to control at least one medical, ophthalmic and optometry equipment; and wherein the one or more controllable medical ophthalmic equipment comprises at least one auto-refractor, digital phoropter, exam table unit; and wherein the equipment are used to administer an vision and eye examination to an patient in a different geographic location from the remote controller.

10. The system of claim 1, wherein at least one equipment, device, and instrument is configured to run and execute at least one equipment control software via one or more user interfaces; and wherein at least one equipment control software is configured to at least an receive command for operating at least one: medical diagnosing equipment, optical measuring device, robotic device, virtual reality headset, telepresence robot, digital phoropter, variable liquid lens phoropter, augmented reality headset, pupillary distance meter and digital focometer, and Internet of things devices sensors; and wherein the one or more equipment control software's are controllable by at least one remote controllers' portable electronic device (PED).

11. The system of claim 1, wherein at least one or more wireless internet networks connection are needed to establish remote administration tool interconnection; and wherein control from second electronic device to first electronic device comprises LAN connection such as Bluetooth, wired, and WIFI; and wherein the PED to MCB connection comprises at least one of: cellphone wireless networks, and Satellite internet connectivity networks to remotely control the MCB that controls the one or more medical and non-medical equipment at the patient location.

12. The system of claim 1, wherein at least one mobile application and software are executed by at least one electronic device; and wherein the electronic device comprises use of at least one of: a remote administration tool, emulation software, emulating software, virtualization software, smart hub, demotics, simulation software, screen share, screen control, speech recognition software, speech synthesis and remote access software.

13. The system of claim 1, wherein coupling, pairing, and connecting from at least one (MCB) second electronic device and at least one first electronic device comprises emulation controlling and viewing of first electronic device interface via second electronic device interface; and wherein the telepresence robot used for eye examination can be a electronic device.

14. The system of claim 1, wherein controlling from a remote controllers' third electronic device (PED), comprises at least one (MCB) second electronic device and telepresence robot equipment to control the control software's of at least one: eye health device, vision examination device, vision screening devices and temperature monitoring device; and wherein the MCB computing device control comprises the remote controller PED to control at least one medical device and one non-medical device from a distance, remote, near, same and different geographic location.

15. (canceled)

16. The system of claim 1, wherein a remote controllers' PED comprises a connection to navigate and control the second electronic device MCB and telepresence robot; and wherein one or more remote controllers user interfaces, including a PED, the PED including at least one mobile application and software's; and wherein the RAT connection comprises an end to end encrypted (HIPPAA) Health Insurance Portability and Accountability Act compliant (remote administration tool) connection and authentication.

17. (canceled)

18. The system of claim 1, wherein controlling the second electronic device MCB interface via a third electronic device (PED) interface to remotely to control one or more software's comprising, emulated screens, control software's and user interfaces dashboard running that control at least one first electronic device that control the control software that controls the ophthalmic equipment, robotic equipment and telecommunication equipment.

19. (canceled)

20. (canceled)

21. The system of claim 1, wherein connect, navigate, and control the telepresence robot to position the vision acuity display screen to presenting optotypes, astigmatism clock dial, Color vision plates, dua-chrome image, optotypes calibration setup, and instructing the person via separated distance range to view and speak the optotypes.

22. (canceled)

23. (canceled)

24. (canceled)

25. The system of claim 1, wherein at least one remote controller's electronic device (PED) is running a mobile application directed to connect via internet protocols to at least one main control base computer system; and wherein the selection, view, control of the TREES comprises at least one the first electronic device that may run a mobile application and software; and wherein the TREES can control at least one of a: phoropter, digital phoropter, variable lens phoropter and digital focometer; and wherein the control, comprises calibrating, navigating and displaying a vision chart software's optotypes via mobile application and software on the MCB and telepresence display screen; and wherein optotypes, lens clock, and dua-chrome screen are presented to determine the sphere power lens, cylinder power lens, and axis of the patient; and wherein refractive services are performed by a PED operated by remote controller from one or more location; and wherein eyeglasses prescription, vision screening report and medical referral form are delivered to the patient via remote means.

26. a method of using a telepresence robot to perform vision examination and eye health examination to a patient, the method comprising: connecting, navigating, and controlling at least one telepresence robot and MCB via a third electronic device PED interface from any geographic location; and delegating movement commands to the telepresence robot to the area requesting service; and administering an eye health and vision exam services to at least one patient; and connecting and determining, the second electronic device computer system MCB user interface at the location; and
instructing the Telepresence robot device commands based on the received user location and data; and wherein examining at least one of the pluralities of patients with the Telepresence robot device including remotely viewing and controlling via at least one camera of the Telepresence robot device and MCB; and maneuvering the telepresence robot to desired location to begin to administer an eye health examination and vision examinations to at least one of a plurality of patients.
coupling, pairing, and connecting from at least one (MCB) second electronic device and at least one first electronic device of the medical device and non-medical device; and
controlling from a third electronic device PED interface at least one (MCB) second electronic device interface and telepresence robot interface to operate at least one medical device and one non-medical device; and
emulating, navigating, and controlling at least one first electronic device equipment control software interface from a second electronic device MCB interface; and
connecting, navigating, and controlling the second electronic device (MCB) interface and telepresence robot interface via one or more networks; and
connecting, navigating, and controlling at least one telepresence robot and main control base interface via a third electronic device (PED) interface and one or more software's interfaces; and
controlling the second electronic device interface via a third electronic device (PED) interface; and
connecting, navigating, and controlling patients' medical history and data via at least one computing device interface and cloud storage; and
controlling, navigating, and viewing the (MCB) interface remotely to control and access patients' data, EMR, equipment control software's interfaces; and
connecting, navigating, and controlling telepresence robot; and
connecting, navigating, controlling, and initiating a visual acuity, vision examination and eye health examination test to a patient using at least one of the medical devices coupled to the MCB and Telepresence robot; and
administering vision acuity, objective refraction test and subjective refraction test to the patient via at least one of: a vision chart, a auto-refractor, a digital phoropter; and
wherein the TREES is remotely controlled by the remote controllers PED interface; and
administering an vision exam and gather a finalized prescription for a patient; and
accessing, controlling, and viewing the (MCB) and telepresence robot electronic device user interface to present information to the patient; and
transmitting, dispensing a medical results, vision screening report, and new eyeglasses prescription to the patient via display and printer connected to the Telepresence robot and MCB.

27. (canceled)

28. The method of claim 26, wherein emulating, navigating and controlling at least one first electronic device medical device control software from a second electronic device MCB; and wherein the remotely controlling at least one user interface of one first electronic device computer system associated to the telepresence robot to perform a remote vision and eye examination to an patient.

29. The method of claim 26, wherein emulating, navigating and controlling at least one first electronic computing device associated with at least one medical device equipment control software interface; and wherein at least one second electronic computing device user interface is able to receive control commands from one or more locations; and wherein controlling at least one or more user interfaces of one or more electronic device computer system to perform a remote Telepresence robot vision and eye examination to an patient.

30. The method of claim 26, wherein controlling, navigating and viewing the one or more user interfaces remotely to control and access user data, EMR, Cloud, equipment control software; and wherein authentication system comprise at least one of: public and private cryptographic keys; and wherein health care providers prescription may use Transaction Hash.

31. The method of claim 26, wherein connecting, navigating, controlling the telepresence robot comprises initiating a visual acuity test to an patient and executing a visual chart software to display optotypes; and providing real-time or near real-time feedback to the patient vision acuity of one and both eyes; and wherein the vision acuity examination includes the patient with and without eyeglasses correction; and wherein response of the examination may comprise at least one of: voice call, video call, speech to text, text to speech, and interactive mobile application.

32. The method of claim 26, wherein accessing, controlling and viewing the (MCB) and telepresence robot electronic device user interface comprises remotely accessing at least one software, and/or medical device control software; and wherein the medical device software controls at least one ophthalmic equipment; and wherein at least one emulated software controller, iriscope, digital pupil-meter, auto refractor, infrared auto-thermometer, digital phoropter, digital optometer and digital focometer to perform telepresence robot eye examination and subjective refraction to an patient.

33. The method of claim 26, wherein connecting, navigating and controlling at least one telepresence robot and main control base via a remote controllers third electronic device to communication with delegate movement commands; and controlling at least one equipment software and eye examination software to performs an eye health and vision screening services to at least one patient in from multiple geographic location.

34. A system for performing remote controlled vision and eye examination to a patient using telepresence robot coupled to one or more ophthalmic medical devices and one or more computing devices, the system comprising:
a first location, including a telepresence robot; and
wherein the telepresence robot, includes coupling to one or more computing device comprising one or more interface controllers; and
wherein the one or more computing device are coupled to one or more ophthalmic medical devices; and
wherein the one or more computing device emulate one or more interfaces onto one or more interface remote controllers computing device interfaces; and
wherein at least one remote controller computing device interface can control and view the telepresence robot computing device interface over one or more networks; and
wherein one or more remote controller computing device are capable control, view and execute command to one or more networks operate at least one of: telepresence robot, computing devices to control one or more medical devices from multiple location to administer one or more vision and eye examination to a patient independent of location; and
wherein the vision and eye examination performed to a patient at the independent location comprise at least one of a real time remote controlled vision acuity, objective refraction, and a subjective refraction examination.
